(19) United States
(12) Patent Application Publication
(10) Pub. No.: US 2016/0289301 A1
(43) Pub. Date: Oct. 6, 2016

(54) PROCESS OF CLONING AND FURTHER PURIFICATION TO MAKE A RECOMBINANT INTRAVENOUS IMMUNOGLOBULIN

(71) Applicant: Kieu Hoang, Agoura Hills, CA (US)

(72) Inventor: Kieu Hoang, Agoura Hills, CA (US)

(21) Appl. No.: 15/090,041

(22) Filed: Apr. 4, 2016

Related U.S. Application Data
(60) Provisional application No. 62/142,237, filed on Apr. 2, 2015.

(51) Int. Cl. C07K 16/08; C07K 16/00

(52) U.S. Cl. CPC C07K 16/082; C07K 16/00; C07K 2317/14; A61K 2039/54

(57) ABSTRACT

The present subject matter is directed to a process of cloning and purifying recombinant intravenous immunoglobulin (IVIG), comprising cloning a target gene of human immunoglobulin; in vitro screening of a yeast cell expressing the target gene of human immunoglobulin to create a yeast cell line; fermenting the yeast cell line and collecting a resulting culture medium; filtering the culture medium; undergoing weak anion exchange chromatography to collect a flow-through solution; ultra-filtrating the flow-through solution to reach a desired protein concentration; aseptic filtrating the flow-through solution; nano filtrating the flow-through solution for virus removal; and filling and incubating the flow-through solution at low pH for virus inactivation to obtain a purified recombinant IVIG. The present subject matter is directed to purified recombinant IVIG having five newly-found proteins, namely KH 33, KH 34, KH 35, KH 36, and KH 37 for both liquid and lyophilized forms.

### RELATED APPLICATIONS

The present patent application claims priority to provisional U.S. Patent Application No. 62/142,237 filed Apr. 2, 2015, which was filed by the inventor hereof and are incorporated by reference herein in its entirety.

### TECHNICAL FIELD

The present subject matter relates to recombinant intravenous immunoglobulin (IVIG) and protein sequences. In particular, the present subject matter is associated with the process of cloning and purifying IVIG to produce purified recombinant IVIG, which contains five newly-found proteins, namely KH 33, KH 34, KH 35, KH 36, and KH 37 in both liquid and lyophilized forms.

### BACKGROUND

Immunoglobulin G (IgG) is a type of antibody and is a protein complex composed of four peptide chains—two identical heavy chains and two identical light chains arranged in a Y-shape typical of antibody monomers. Each IgG has two antigen binding sites. Representing approximately 75% of serum antibodies in humans, IgG is the most common type of antibody found in the circulation. IgG molecules are created and released by plasma B cells. Antibodies are major components of humoral immunity. IgG is the main type of antibody found in blood and extracellular fluid, allowing it to control infection of body tissues. IgG protects the body from infection by binding many kinds of pathogens, such as viruses, bacteria, and fungi. In the plasma-derived industry, IgG is purified from human plasma from Fraction II. However, a certain percentage of IgG is precipitated into Fraction III paste.

In a related application, the Applicant has shown IVIG, in vitro and in vivo, having 7 existing and newly-found proteins may cure and prevent Hepatitis B virus infection. The limited quantity of IVIG produced from plasma will not be enough to provide treatment for the at least 350,000,000 HBV carriers around the globe, not to mention the at least 130,000,000 HBV carriers in China alone. Only 250,000 vials can be produced from 1,000,000 liters of WIG, which may be used to cure 25,000 patients. Thus, less than 0.00714% of patients may be cured, leading to a demand for the present subject matter.

The present subject matter is directed to cloning of the total of 7 existing and newly-found proteins, and then expressing individual proteins using different strains of yeast to obtain the maximum of at least 5,000,000,000 (5 billion cells per ml). Through processing culture medium, the targeted protein may be purified. From the process, either one final targeted protein can be obtained or seven individual targeted proteins are combined to make a recombinant IVIG.

### SUMMARY

In an embodiment of the present subject matter, recombinant DNA technology is used to clone five newly-found proteins or two existing light chains and heavy chains in immunoglobulin G for intravenous injection. The five cloned newly-found proteins are expressed in yeast cells. With these newly-found proteins, the recombinant intravenous solution of immunoglobulin stops replication of Hepatitis B virus and also prevents Hepatitis B virus infection. A recombinant WIG with the newly-found or existing proteins performs the same functions as plasma-derived intravenous immunoglobulin.

In an embodiment, the present subject matter is directed to a process of cloning and purifying recombinant intravenous immunoglobulin (IVIG), comprising the steps:
a) cloning a target gene of human immunoglobulin;
b) in vitro screening of a yeast cell expressing the target gene of human immunoglobulin to create a yeast cell line;
c) fermenting the yeast cell line and collecting a resulting culture medium;
d) filtering the culture medium with 10CP+90SP;
e) undergoing weak anion exchange chromatography to collect a flow-through solution;
f) ultra-filtrating the flow-through solution to reach a desired protein concentration;
g) aseptic filtrating the flow-through solution;
h) nano filtrating the flow-through solution for virus removal with a 20 nm filter; and
i) filling and incubating the flow-through solution at low pH for virus inactivation to obtain a purified recombinant IVIG.

### BRIEF DESCRIPTION OF THE DRAWINGS

FIG. 1 is a flow chart depicting a method of processing recombinant IVIG.

FIG. 2 is a photograph illustrating the SDS-PAGE analysis of newly-found proteins KH 33, KH 34, KH 35, KH 36, and KH 37. The SDS-PAGE analysis shows the expression of newly-found proteins individually and that the molecular weights are close to the molecular weights of the two main proteins in IVIG, namely the heavy chain and light chain of IVIG.

FIG. 3 is a photograph illustrating that by using a dual DNA expressing system, KH 33 and KH 37 have been expressed in one yeast cell. KH 33 represents the light chain of IVIG, while KH 37 represents the heavy chain of IVIG.

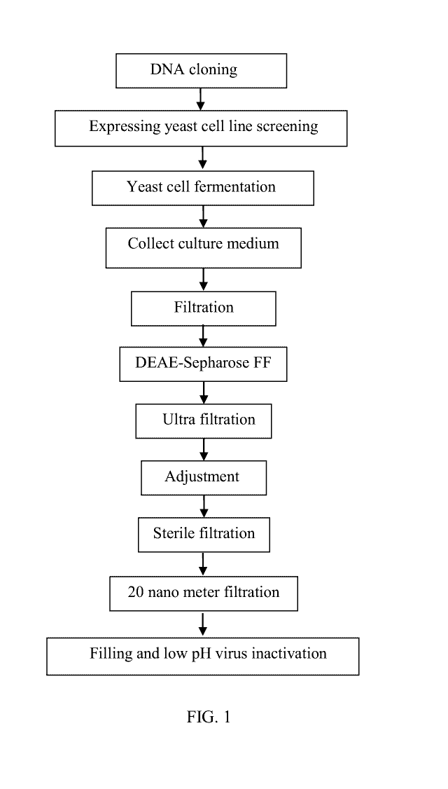

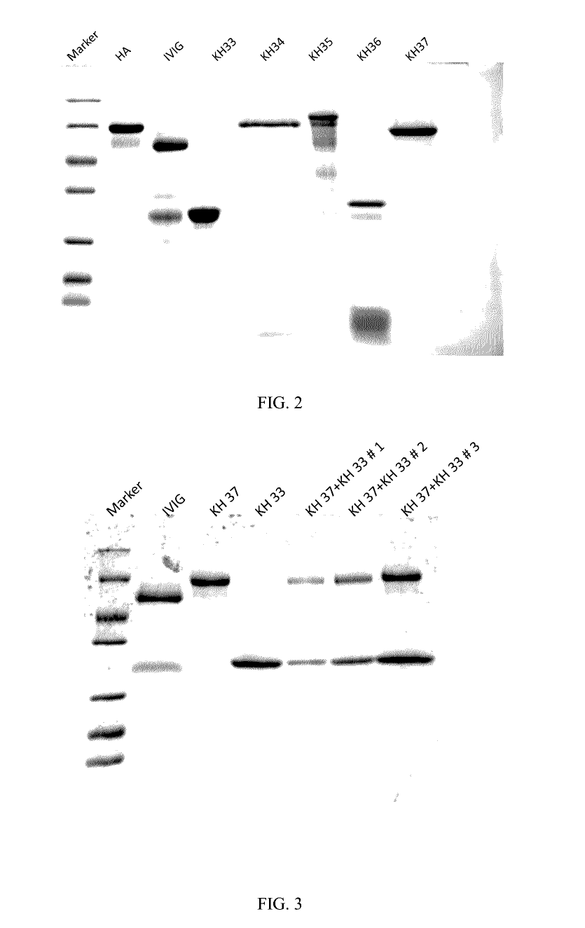

### DETAILED DESCRIPTION

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

An embodiment of the present subject matter is directed to a process of cloning and purifying recombinant intravenous immunoglobulin (IVIG), comprising the steps:
a) cloning a target gene of human immunoglobulin;
b) in vitro screening of a yeast cell expressing the target gene of human immunoglobulin to create a yeast cell line;
c) fermenting the yeast cell line and collecting a resulting culture medium;
d) filtering the culture medium with 10CP+90SP;
e) undergoing weak anion exchange chromatography to collect a flow-through solution;
f) ultra-filtrating the flow-through solution to reach a desired protein concentration;
g) aseptic filtrating the flow-through solution;
h) nano filtrating the flow-through solution for virus removal with a 20 nm filter; and
i) filling and incubating the flow-through solution at low pH for virus inactivation to obtain a purified recombinant IVIG.

In an embodiment, the purified recombinant IVIG comprises proteins KH 33, KH 34, KH 35, KH 36, and KH 37. The purified recombinant may be in liquid or lyophilized form. Further, an embodiment of the present subject matter is directed to a purified recombinant IVIG produced according to the process of cloning and purifying recombinant IVIG.

In an embodiment, the target gene of human immunoglobulin is found and sequenced in plasma-derived IVIG. The process may further comprise transfecting IVIG gene sequences into the yeast cell line to obtain a yeast cell line expressing IVIG and screening the yeast cell line expressing IVIG with a dual antibiotic marker. The process may further comprise expanding a yeast cell line expressing IVIG and culturing in a bioreactor to maximize cell numbers to at least 5 billion cells per ml.

In an embodiment, the weak anion exchange chromatography is conducted using DEAE sepharose Fast Flow (DEAE sepharose FF), a chromatograph medium. In an embodiment, the flow-through solution is ultra filtered with a 10 K cutoff membrane. The process may further comprise adjusting the protein concentration and pH of the flow-through solution. In an embodiment, the aseptic filtration is 0.22 μm aseptic filtration. In an embodiment, the flow-through solution is subjected to filling and low pH incubation at pH 4 for 21 days at 25° C. as the virus inactivation.

An embodiment of the present subject matter is directed to a method of stopping replication of a Hepatitis B virus in a patient comprising administering the purified recombinant IVIG obtained from the process of cloning and purifying recombinant IVIG to a patient in need thereof. An embodiment of the present subject matter is directed to a method of killing a Hepatitis B virus in a patient comprising administering the purified recombinant IVIG obtained from the process of cloning and purifying recombinant WIG to a patient in need thereof. An embodiment of the present subject matter is directed to a method of preventing infection of a Hepatitis B virus in a patient comprising administering the purified recombinant IVIG obtained from the process of cloning and purifying recombinant IVIG to a patient in need thereof.

Furthermore, an embodiment of the present subject matter is directed to a method of preventing one or more of H1N1 and other bird flu virus infections in a patient comprising administering the purified recombinant IVIG obtained from the process of cloning and purifying recombinant IVIG to a patient in need thereof.

In an embodiment, the present subject matter is directed to a method of treating a patient in need thereof comprising administering the purified recombinant IVIG obtained from the process of cloning and purifying recombinant IVIG to the patient, wherein the purified recombinant IVIG transforms or repairs damaged and sick cells to become healthy cells, wherein the purified recombinant IVIG protects cellular alterations, and wherein the purified recombinant IVIG signals a body to produce new cells that are healthy, thereby preventing the new cells from being affected by intracellular and extracellular damaging signals.

### EXAMPLES

According to an embodiment of the present subject matter, . . .

### Testimonials

An embodiment . . .

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

I claim:

1. A process of cloning and purifying recombinant intravenous immunoglobulin (IVIG), comprising the steps:
a) cloning a target gene of human immunoglobulin;
b) in vitro screening of a yeast cell expressing the target gene of human immunoglobulin to create a yeast cell line;
c) fermenting the yeast cell line and collecting a resulting culture medium;
d) filtering the culture medium with 10CP+90SP;
e) undergoing weak anion exchange chromatography to collect a flow-through solution;
f) ultra-filtrating the flow-through solution to reach a desired protein concentration;
g) aseptic filtrating the flow-through solution;
h) nano filtrating the flow-through solution for virus removal with a 20 nm filter; and
i) filling and incubating the flow-through solution at low pH for virus inactivation to obtain a purified recombinant IVIG.

2. The process of claim 1, wherein the resulting purified recombinant IVIG comprises proteins KH 33, KH 34, KH 35, KH 36, and KH 37.

3. The process of claim 1, wherein the target gene of human immunoglobulin is found and sequenced in plasma-derived IVIG.

4. The process of claim 1, further comprising transfecting IVIG gene sequences into the yeast cell line to obtain a yeast cell line expressing IVIG and screening the yeast cell line expressing IVIG with a dual antibiotic marker.

5. The process of claim 4, wherein the yeast cell line expressing IVIG is expanded and cultured in a bioreactor to maximize cell numbers to at least 5 billion cells per ml.

6. The process of claim 1, wherein the weak anion exchange chromatography is DEAE sepharose FF.

7. The process of claim 1, wherein the flow-through solution is ultra filtered with a 10 K cutoff membrane.

8. The process of claim 1, further comprising adjusting the protein concentration and pH of the flow-through solution.

9. The process of claim 1, wherein the aseptic filtration is 0.22 μm aseptic filtration.

10. The process of claim 1, wherein the flow-through solution is subjected to filling and low pH incubation at pH 4 for 21 days at 25° C. as the virus inactivation.

11. A method of stopping replication of a Hepatitis B virus in a patient comprising administering the purified recombinant IVIG obtained from the process of claim 1 to a patient in need thereof.

12. A method of killing a Hepatitis B virus in a patient comprising administering the purified recombinant IVIG obtained from the process of claim 1 to a patient in need thereof.

13. A method of preventing infection of a Hepatitis B virus in a patient comprising administering the purified recombinant IVIG obtained from the process of claim 1 to a patient in need thereof.

14. A method of preventing one or more of H1N1 and other bird flu virus infections in a patient comprising administering the purified recombinant IVIG obtained from the process of claim 1 to a patient in need thereof.

15. A purified recombinant IVIG produced according to the process of claim 1.

16. A method of treating a patient in need thereof comprising administering the purified recombinant IVIG obtained from the process of claim 1 to the patient,
wherein the purified recombinant IVIG transforms or repairs damaged and sick cells to become healthy cells,
wherein the purified recombinant WIG protects cellular alterations, and
wherein the purified recombinant WIG signals a body to produce new cells that are healthy, thereby preventing the new cells from being affected by intracellular and extracellular damaging signals.

17. The purified recombinant WIG of claim 1, wherein the purified recombinant IVIG is in liquid form.

18. The purified recombinant WIG of claim 1, wherein the purified recombinant IVIG is in lyophilized form.
